(19) United States
(12) Patent Application Publication
(10) Pub. No.: US 2026/0193011 A1
(43) Pub. Date: Jul. 9, 2026

(54) MULTI-CHAMBERED DISPENSING CAPSULE

(71) Applicant: TRISTEL PLC, Newmarket (GB)

(72) Inventors: Jeremy TURNER, Newmarket (GB); Elanor DIXON, Newmarket (GB); Paul SWINNEY, Newmarket (GB)

(21) Appl. No.: 19/130,757

(22) PCT Filed: Nov. 16, 2023
(86) PCT No.: PCT/GB2023/053020

§ 371 Date: May 16, 2025

(30) Foreign Application Priority Data
Nov. 17, 2022 (GB) 2217208.4

(51) Int. Cl. B65D 81/32

(52) U.S. Cl. CPC B65D 81/3294

(57) ABSTRACT

A dispensing capsule comprises a capsule body having an end face, a first chamber and a second chamber, the chambers being defined by respective first and second collapsible walls of the capsule body, the first and second chambers opening on to the end face of the capsule body at respective first and second openings and being closed by respective first and second base walls. A seal member can be applied to the end face of the capsule body to close the first and second chambers. The end face comprises a first sealing rib that circumscribes the first opening and a second sealing rib that circumscribes the second opening, the seal member being sealable to the first and second sealing ribs when applied to the end face.

### FIELD OF THE INVENTION

The present invention relates to a multi-chambered dispensing capsule, in particular, but not exclusively, for containing decontaminating compositions and components of two-part decontaminating compositions.

### BACKGROUND TO THE INVENTION

Many liquid preparations include active ingredients which degrade over time, limiting product shelf life. This is particularly true for disinfectants or sterilising agents such as chlorine dioxide, where the active ingredient is formed in situ when required by mixing two reagents. Examples are disclosed in WO 2005/011756. Chlorine dioxide, for example, may be formed by mixing a chlorite solution and an acid.

WO 2017/060677 discloses a dispensing capsule having a body that provides two chambers or cavities, separated from one another by a dividing wall. Each chamber is defined by a collapsible wall and has an opening at a first end of the body of the capsule. At a second end, opposite the first end, a closed end of each chamber carries a burst pin that extends towards the first end. A peripheral wall extends around the periphery of the first end of the body. A flexible seal is bonded to the peripheral wall and to the dividing wall to seal each of the first and second chambers. The seal is bonded less strongly to the dividing wall than to the peripheral wall. The contents of the capsule can be dispensed by applying a force to the collapsible walls from the second end of the capsule to increase the pressure in the chambers. Initially, this causes the seal to detach from the dividing wall, leaving the bond between the seal and the peripheral wall intact. This allows the contents of the two chambers to start to mix while still contained by the seal. Subsequently, the burst pins contact the seal and help to break the bond between the seal and the peripheral wall, releasing the contents of the capsule.

Capsules of the type described in WO 2017/060677 can beneficially be used for in-situ generation of disinfecting compositions such as chlorine dioxide, where each chamber contains one of the two parts or reagents required to form the active ingredient in a concentrated form. Such capsules may be arranged to dispense their contents into a diluent (typically water), contained within a primary container. For example, the primary container may be a bottle, and the capsule may be arranged to engage with the neck of the bottle. In such cases, a screw-on cap including a plunger may be provided to apply the force to the walls of the chambers, as described in WO 2017/060677. WO 2019/135065 describes a similar bottle arrangement in which the capsule dispenses its contents into a cup member to allow the concentrated reagents to mix for longer before release into the diluent.

The above-described capsules are convenient for applications in which pre-mixing within the capsule is desired. However, in other applications, pre-mixing within the capsule may not be required, and in such cases it may be desirable to provide a dispensing capsule with two or more chambers in which the likelihood of mixing of the contents of the chambers upon mechanical shock or prolonged storage is reduced. It is against that background that the present invention has been devised.

### SUMMARY OF THE INVENTION

Aspects of the invention are specified in the independent claims. Preferred features are specified in the dependent claims.

A dispensing capsule according to one embodiment of the invention comprises a capsule body having a first end and a second end, the first end providing an end face of the capsule body, a first chamber, and a second chamber, the chambers being defined by respective first and second collapsible walls of the capsule body. The first and second chambers open on to the end face of the capsule body at respective first and second openings and are closed by respective first and second base walls arranged parallel with the second end of the capsule body. The dispensing capsule also comprises a seal member for application to the end face of the capsule body, thereby to close the first and second chambers, wherein, in use, the first and second collapsible walls collapse towards the end face upon application of a force to the first and second base walls from the second end of the capsule body and directed towards the first end, thereby to cause the seal member to unseal the first and second chambers.

The end face comprises a first sealing rib that circumscribes the first opening and a second sealing rib that circumscribes the second opening, the seal member being sealable to the first and second sealing ribs when applied to the end face. The first and second sealing ribs are unconnected to one another and define an uninterrupted gap that spans across the end face of the capsule between the first and second openings.

With this arrangement, when the seal member is applied, the seal lines formed between the seal member and the first and second sealing ribs do not intersect, connect or overlap. This reduces the likelihood of leakage from one of the chambers reaching the other chamber, causing premature mixing of the contents of the two chambers. For example, in the case of a mechanical impact resulting in the failure of one of the seal lines, there is a reduced risk of mixing of the contents of the two chambers. Premature mixing of the contents of the two chambers may be undesirable, for example in cases where the two chambers contain reagents that react to form an active ingredient that requires handling precautions, and/or where the active ingredient will degrade over time to render it ineffective. Both of these factors may be present where the reagents form a high-level disinfecting composition such as chlorine dioxide.

It has also been observed that, in prior art arrangements in which chambers are separated by a single seal line, or where the seal line of one chamber is continuous with the seal line of another chamber, the contents of one or both of the chambers can migrate over time across the seal lines by capillary flow and can therefore mix prematurely with one another. The present invention significantly reduces this possibility.

In this context, therefore, the first and second sealing ribs are unconnected to one another in the sense that the first and second sealing ribs do not intersect, overlap, join, merge or otherwise connect, and that there are no intermediate structures that extend between the first and second sealing ribs in a way that would result in a first seal line formed between the first sealing rib and the seal member being connected to or continuous with a second seal line formed between the second sealing rib and the seal member.

Preferably, when the seal member is applied to the end face, each of the first and second sealing ribs has an inner side that is entirely within a sealed volume corresponding to the respective first or second chamber, and an outer side that is entirely outside said sealed volume. The uninterrupted gap is preferably open to an outside of the dispensing capsule. In these ways, any leakage from the first chamber or the second chamber is not captured within the capsule, but instead is released externally and can therefore be readily detected by a user.

In some embodiments, each of the first and second base walls comprises a burst pin that extends into the corresponding chamber and towards the first end of the capsule body, each burst pin being arranged to rupture the seal member to unseal the corresponding first or second chamber upon collapse of the first and second collapsible walls. Each burst bin may comprise a free end having at least one pointed projection. For example, the free end of each burst pin may have a pair of pointed projections. Each burst pin may have a generally H-shaped cross-section in a plane parallel to the end face, to increase the stiffness of the burst pin.

The seal member may be bonded to the first and second sealing ridges by any suitable means, for example by an adhesive or by heat sealing.

Preferably, a bond strength between the seal member and the first and second sealing ridges is sufficient for the seal member to rupture before the seal member unseals from the sealing ridges during application of the force.

The capsule may be adapted for use with a vessel, such as a bottle, where the capsule can be located in the neck of the bottle and actuated by a plunger provided on a cap of the bottle. The bottle may contain a diluent. In other arrangements, the diluent is held in a tank with a dispensing tap, or a jug with a spout, and in both such cases the capsule may be inserted into a capsule-receiving part of a lid or cover. Here, a plunger may be provided on a pivoting lever attached to the lid.

### BRIEF DESCRIPTION OF THE DRAWINGS

The invention will now be further described, by way of example only, with reference to the following drawings in which like reference signs are used for like features, and in which:

FIG. 1 is an exploded perspective view from a first end of a dispensing capsule with a seal member;

FIG. 2 is a first end view of the dispensing capsule of FIG. 1, with the seal member omitted;

FIG. 3 is a sectional view of the dispensing capsule of FIG. 1, taken on line A-A in FIG. 2 and with the seal member omitted;

FIG. 4 is a second end view of the dispensing capsule of FIG. 1;

FIG. 5 is a perspective view from a second end of the dispensing capsule of FIG. 1;

FIG. 6 is a first end perspective view of the dispensing capsule of FIG. 1 with the seal member in place;

FIG. 7 shows seal lines on the seal member;

FIG. 8(a) to 8(c) show a sequence of steps in the use of the dispensing capsule of FIG. 1 to dispense the contents of the capsule; and

FIG. 9 shows a dispensing capsule in use in an apparatus for preparing a liquid preparation.

### DETAILED DESCRIPTION

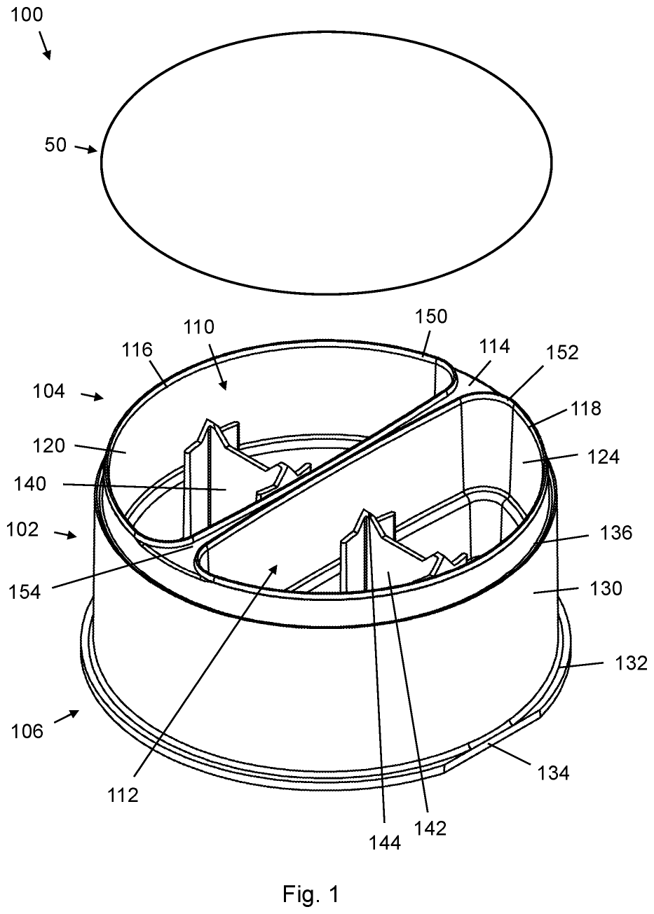

FIG. 1 is an exploded view of a dispensing capsule 100 according to an embodiment of the invention. The capsule 100 comprises a capsule body 102 having a first end 104 (uppermost in FIG. 1) and a second end 106 (lowermost in FIG. 1). A seal member 50, formed from a flexible thin sheet material, is attachable to the first end 104 of the capsule body, as will be described in more detail below. In FIG. 1, the seal member 50 is shown spaced away from the capsule body 102 to reveal parts of the interior of the capsule body 102.

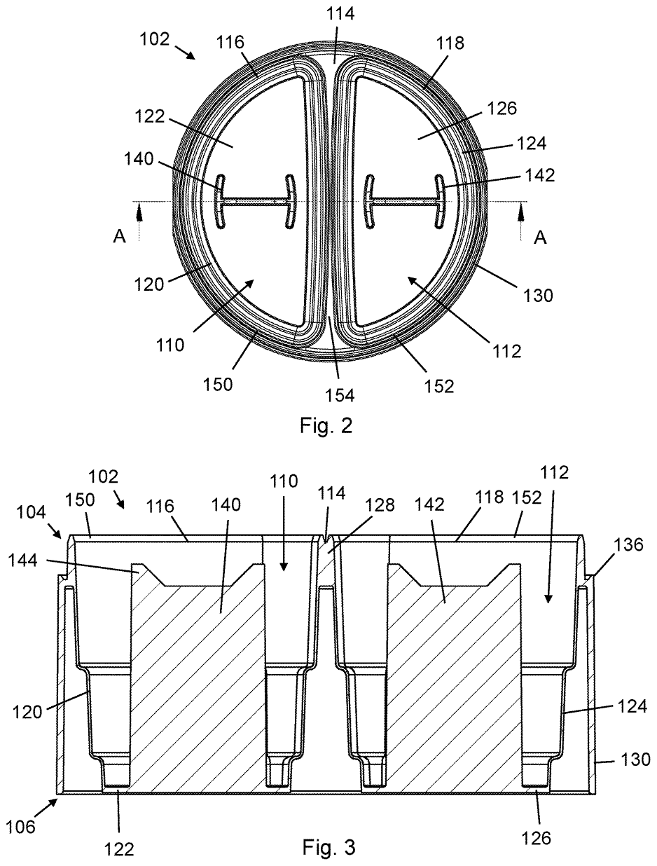

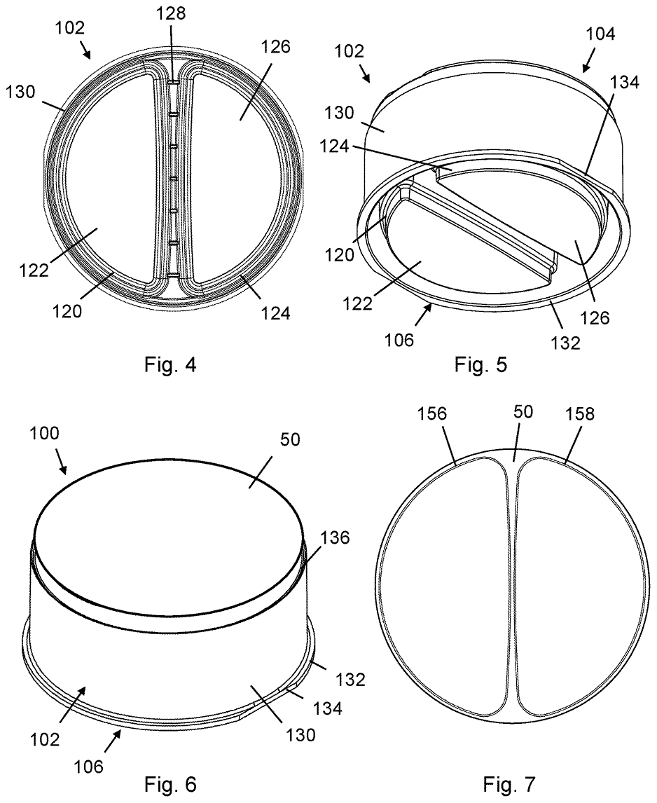

Referring additionally to FIGS. 2 and 3, the capsule body 102 is shaped to form a first cavity or chamber 110 and a second cavity or chamber 112. Each chamber 110, 112 opens onto an end face 114 provided at the first end 104 of the capsule body 102, to provide respective first and second openings 116, 118 on the end face 114. The first chamber 110 is defined by a first wall 120 that extends from the end face 114 towards the second end 106 of the capsule body 102. The wall 120 extends around the first chamber 110 to form a generally vertical side wall of the chamber 110. As shown most clearly in FIG. 3, and with reference also to FIGS. 4 and 5, the first chamber 110 is closed by a first base wall 122 disposed parallel to and aligned with the second end 106 of the capsule body 102. In the same way, the second chamber 112 is defined by a second wall 124 that extends from the end face 114 and a second base wall 126.

In this example, the first and second walls 120, 124 are configured to collapse in a telescoping fashion. To this end, and referring in particular to FIG. 3, each of the first and second walls 120, 124 includes three connected sections of decreasing peripheral length moving toward the respective base wall 122, 126, so that each of the first and second chambers 110, 112 has three sections of decreasing lateral dimensions moving towards the second end of the capsule body 102.

Referring to FIGS. 3 and 4, where the first and second walls 120, 124 lie adjacent to one another along a centreline of the capsule body 102, a plurality of stiffening ribs 128 extend between the first and second walls 120, 124 and connect with the end face 114. The stiffening ribs 128 help to reduce bending or flexing of the capsule body 102 along the centreline between the chambers 110, 112.

The capsule body 102 has a peripheral outer wall 130 that extends from the periphery of the end face 114 and extends towards the second end 106 of the capsule body 102. The capsule body 102 is open at is second end 106 so that the first and second base walls 122, 126 are accessible from an opening formed by the free end of the outer wall 130 (see FIGS. 4 and 5).

The outer wall 130 is provided with a flange 132 at the second end 106 of the capsule body 102, which stiffens the outer wall 130. The flange 132 is provided with orientation flats 134 for use during manufacturing and filling of the dispensing capsule 100. Referring again to FIGS. 1 and 3, adjacent to but spaced from the first end 104 of the capsule body 102, the outer wall 130 is shaped to provide an annular locating shoulder 136 having a ridge that projects towards the first end 104. In use, the locating shoulder 136 and/or the flange 132 can be used to support the dispensing capsule 102 in a receiving region of a container, as will be described further below.

A first burst pin 140 is disposed in the first chamber 110, and a second burst pin 142 is disposed in the second chamber 112. Each burst pin 140, 142 is attached to the corresponding first or second base wall 112, 126, and extends towards the first end 104 of the capsule body 102. As shown most clearly in FIG. 1, the free end of each burst pin 140, 142 carries a pair of pointed projections 144. The tip of each of the projections 144 is spaced from the first end 104 of the capsule body 102, as can be seen in FIG. 3. Referring to FIG. 2, the cross-sectional shape of each burst pin 140, 142, viewed in a plane parallel to the end face 114, has an “H” shape, to increase the stiffness of the burst pin 140, 142.

The end face 114 of the capsule body 102 comprises a first sealing rib 150 that circumscribes the first opening 116, and a second sealing rib that 152 that circumscribes the second opening 118. In this way, the first sealing rib 150 extends in an unbroken manner around the whole periphery of the first opening 116, and the second sealing rib 152 similarly extends in an unbroken manner around the whole periphery of the second opening 118. Each sealing rib 150, 152 provides a raised surface against which the seal member 50 can be sealed. The first and second sealing ribs 150, 152 are not connected to one another, so that the first and second sealing ribs 150, 152 are spaced apart to define an uninterrupted gap 154 that spans across the end face of the capsule along the centreline between the first and second openings 116, 118.

The dispensing capsule 100 can be filled during manufacture by introducing a first substance into the first chamber 110 and a second substance into the second chamber 112. The first and second substances may be different substances, and in particular may be substances that react together to form an active ingredient. However, it is also possible that the first and second substances are the same substance. The substances are preferably both liquids, although other forms of substance, such as powders, granules, gels, waxes, sols, pastes, aerosols and so on are also possible.

Once the first and second chamber 110, 112 have been filled, the seal member 50 is applied to the end face 114 of the capsule body 102. The seal member 50 is bonded to the first sealing rib 150 and the second sealing rib 152, for example by heat bonding, or by a suitable adhesive. FIG. 7 shows the shape of the resulting first and second sealing lines 156, 158 between the first and second sealing ribs 150, 152 and the seal member 50. Because the first and second sealing ribs 150, 152 are not connected to one another, no part of the first sealing line 156 between the first sealing rib 150 and the seal member 50 intersects with or contacts any part of the second sealing line 158 between the second sealing rib 152 and the seal member 50. In this way, any of the first substance that leaks by capillary migration or otherwise from the first chamber 110 across the first seal line 156 does not reach the second substance in the second chamber 112, and vice versa in the case of leakage of the second substance across the second seal line 158. This substantially reduces the likelihood of undesired mixing of the first and second substances in the event of a partial failure of the seal between the capsule body 102 and the seal member 50, for example due to a mechanical impact, or due to migration across the seal during long-term storage of the filled capsule 100.

Furthermore, it will be appreciated from FIG. 7 that leakage of either the first or the second substance is not contained within the dispensing capsule 100, but instead can reach the outside of the capsule 100. This reduces the risk that a user remains unaware that a dispensing capsule 100 has suffered a failure of the seal, since it will be apparent that a leak has occurred.

The seal member 50 is preferably a sheet or foil material, and suitable materials will be familiar to those skilled in the art. The seal member 50 may, for example, be formed from a laminate of polyethylene (PE)/polyamide (PA)/ethylene-vinyl acetate (EVA) or PE/Aluminium/EVA. The capsule body 102 may be formed from any suitable structural material, for example a plastics material such as LDPE, and may be formed by injection moulding, 3D printing or by any other suitable method. The seal 30 may be bonded to the sealing ribs 150, 152 by any suitable techniques; for example by welding or by means of an adhesive. Suitable adhesives and welding techniques will be known to those skilled in the art.

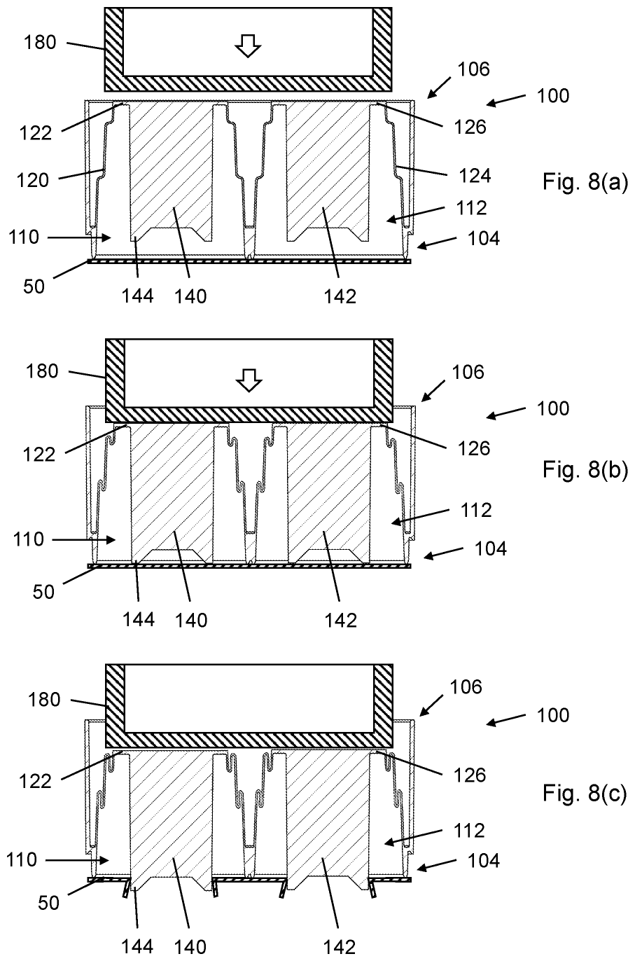

FIG. 8(a) to 8(c) show stages in the use of the dispensing capsule 100. First, as shown in FIG. 8(a), the capsule 100 (with the seal member 50 sealed to the capsule body 102) is placed in a dispensing apparatus, of which only a plunger 180 is shown schematically. The capsule 100 is oriented with the seal member 50 and first end 104 lowermost. Initially, the plunger 180 is spaced from the second end 106 of the capsule.

When it is desired to dispense the contents of the capsule 100, the plunger 180 is brought into contact with the first and second base walls 122, 126 and moved downwards, towards the first end 104 of the capsule body, to apply a force to the first and second base walls 122, 126 from the second end 106 of the capsule body. As shown in FIG. 8(b), this movement of the plunger 180 displaces the first and second base walls 122, 126 towards the first end 104, progressively collapsing the first and second walls 120, 124, and bringing the pointed tips 144 of the burst pins 140, 142 closer to the seal member 50.

Further movement of the plunger 180 further collapses the first and second walls 120, 124, bringing the pointed tips 144 of the burst pins 140, 142 into contact with the seal member 50, thereby piercing the seal member 50 as shown in FIG. 8(c). This releases the contents of both of the chambers 110, 112 substantially simultaneously.

The collapse of the first and second walls 120, 124 reduces the volume of, and therefore increases the pressure in, the first and second chambers 110, 112. However, in the example illustrated in FIG. 8, the seal member 50 remains intact and sealed to the sealing ribs 150, 152 until the burst pins 140, 142 contact the seal member 50, and the chambers 110, 112 are unsealed to release their contents when the seal member 50 is pierced or burst by the burst pins 140, 142.

In other embodiments, the increase in pressure in the chambers 110, 112 may cause the seal member 50 to detach from the sealing ribs 150, 152, releasing the contents of the chambers 110, 112 without piercing or bursting of the seal member 50. In such cases, the burst pins 140, 142 may be arranged to contact the seal member 50 and help to push the seal member away from the sealing ribs 150, 152, in which case the pointed projections 144 could be omitted or replaced with rounded or flat projections. In other cases, the burst pins 140, 142 may be omitted entirely. It is also conceivable that the bond strength between the seal member 50 and the sealing ribs 150 may be sufficient for the seal member 50 itself to rupture or burst without the use of burst pins 140, 142.

The dispensing capsule 100 may be fitted into a receiving region of a suitable container. In one example, the container is a bottle that optionally contains a diluent, and the capsule 100 is received in a neck part of the bottle, supported by the shoulder 136 on a corresponding feature of the neck, with the ridge helping to seal the capsule 100 in the bottle. The plunger 180 may be provided as part of a screw-on or push-on cap of the bottle, so that fitting the cap to the bottle causes the plunger 180 to actuate the dispensing capsule 100, releasing its contents into the bottle. Such arrangements are described in WO 2017/060677 and WO 2019/135065.

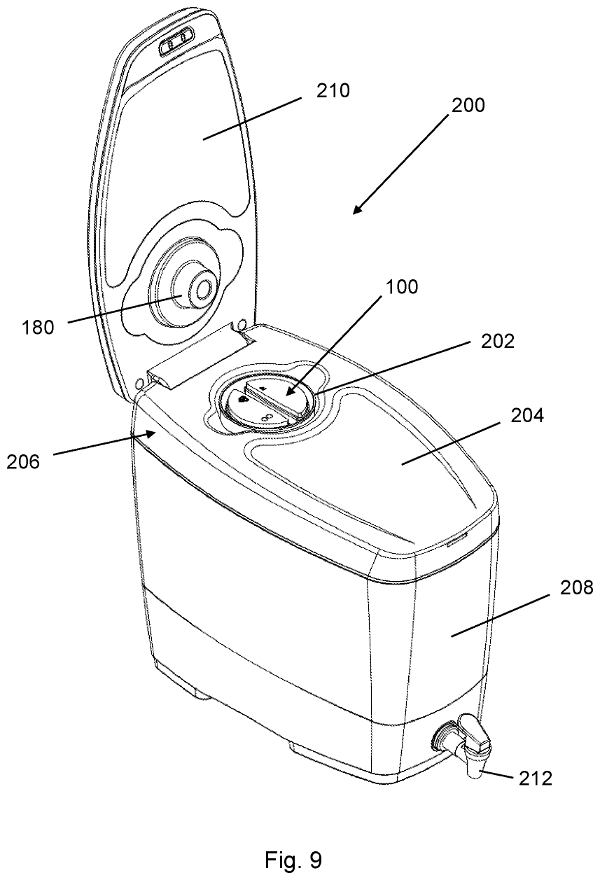

In another example, shown in FIG. 9, a tank apparatus 200 is provided, and the dispensing capsule 100 is received in a receiving region 202 provided in a base part 204 of a lid assembly 206 of the apparatus 200. The lid assembly 206 is disposed above a tank 208 that can be filled with a diluent such as water. The plunger 180 is provided on a lever-like cover 210 of the lid assembly 206 that is connected to the base part 204 at a hinge. The cover 210 can be raised to allow the capsule 100 to be placed in the receiving region 202, and then lowered to actuate the capsule 100 with the plunger 180, releasing the contents of the capsule 100 into the tank 208. The resulting preparation can be dispensed from the apparatus 200 by a tap 212.

It will be understood that dispensing capsules of the type described above can be used with other dispensing apparatus, and that suitable modifications to the capsule can be contemplated for compatibility with such apparatus.

The contents of the capsule may, for example, be reagents which when mixed produce a disinfectant composition, for example chlorine dioxide or peracetic acid. Suitable reagents will be well known to those skilled in the art. For example, reagents for producing chlorine dioxide include: chlorite and acid; chlorate, peroxide and acid; and chlorite, hypochlorite, and a suitable buffer. The reagents may be in a concentrated form, providing rapid formation of the active agent when the contents of the chambers are mixed. To that end, the dispensing apparatus may dispense the contents of the capsule into a mixing cup, funnel or chamber, and subsequently release the mixed substance into a diluent.

It will be appreciated that the capsule body 102 may be of any size appropriate to its intended use. In the illustrated example, each of the first and second chambers 110, 112 is sized to accommodate 100 mL of a liquid substance, for a total capacity of 200 mL. However, the capsule 100 may be larger or smaller. The capsule may include more than two chambers, so that three or more substances can be accommodated.

Further variations and modifications not explicitly described above may also be contemplated without departing from the scope of the invention as defined in the appended claims.

1. A dispensing capsule comprising:
a capsule body having a first end and a second end, the first end providing an end face of the capsule body;
a first chamber and a second chamber, the chambers being defined by respective first and second collapsible walls of the capsule body; the first and second chambers opening on to the end face of the capsule body at respective first and second openings and being closed by respective first and second base walls arranged parallel with the second end of the capsule body; and
a seal member for application to the end face of the capsule body, thereby to close the first and second chambers;
wherein, in use, the first and second collapsible walls collapse towards the end face upon application of a force to the first and second base walls from the second end of the capsule body and directed towards the first end, thereby to cause the seal member to unseal the first and second chambers;
wherein the end face comprises a first sealing rib that circumscribes the first opening and a second sealing rib that circumscribes the second opening, the seal member being sealable to the first and second sealing ribs when applied to the end face;
and wherein the first and second sealing ribs are unconnected to one another and define an uninterrupted gap that spans across the end face of the capsule between the first and second openings.

2. The dispensing capsule of claim 1 wherein, when the seal member is applied to the end face, each of the first and second sealing ribs has an inner side that is entirely within a sealed volume corresponding to the respective first or second chamber, and an outer side that is entirely outside said sealed volume.

3. The dispensing capsule of claim 1, wherein the uninterrupted gap is open to an outside of the dispensing capsule.

4. The dispensing capsule of claim 1, wherein each of the first and second base walls comprises a burst pin that extends into the corresponding first or second chamber and towards the first end of the capsule body, each burst pin being arranged to rupture the seal member to unseal the corresponding first or second chamber upon collapse of the first and second collapsible walls.

5. The dispensing capsule of claim 4, wherein each burst bin comprises a free end having at least one pointed projection.

6. The dispensing capsule of claim 5, wherein the free end of each burst pin has a pair of pointed projections.

7. The dispensing capsule of claim 4, wherein each burst pin has a generally H-shaped cross-section in a plane parallel to the end face.

8. The dispensing capsule of claim 1, wherein the seal member is bonded to the first and second sealing ridges by an adhesive or by heat sealing.

9. The dispensing capsule of claim 1, wherein a bond strength between the seal member and the first and second sealing ridges is sufficient for the seal member to rupture before the seal member unseals from the sealing ridges during application of the force.

10. A dispensing capsule comprising:
a capsule body having a first end and a second end, the first end providing an end face of the capsule body;
a first chamber and a second chamber, the chambers being defined by respective first and second collapsible walls of the capsule body; the first and second chambers opening on to the end face of the capsule body at respective first and second openings and being closed by respective first and second base walls arranged parallel with the second end of the capsule body; and
a seal member for application to the end face of the capsule body, thereby to close the first and second chambers;
wherein, in use, the first and second collapsible walls collapse towards the end face upon application of a force to the first and second base walls from the second end of the capsule body and directed towards the first end, thereby to cause the seal member to unseal the first and second chambers;
wherein the end face comprises a first sealing rib that circumscribes the first opening and a second sealing rib that circumscribes the second opening, the seal member being sealable to the first and second sealing ribs when applied to the end face;
wherein, when the seal member is applied to the end face, each of the first and second sealing ribs has an inner side that is entirely within a sealed volume corresponding to the respective first or second chamber, and an outer side that is entirely outside said sealed volume.
